SEQUENCE LISTING (11 sequences)

SEQ ID NO: 1 - DNA, 173 bp, Artificial Sequence
  conserved sequence 1 on gag gene from HIV-1
ttccctcaaatcactctttggcaacgaccccttgtcacagtaaaaataggaggacagctgaaagaagctctattagatacaggagcagatgatacagtattagaagatataaatttgccaggaaaatggaaaccaaaaatgatagggggaattggaggttttatcaaggtaaa

SEQ ID NO: 2 - DNA, 152 bp, Artificial Sequence
  conserved sequence 2 on gag gene from HIV-1
gcatcagaaggaacctccattcctttggatgggatatgaactccatcctgacagatggacagtccagcctatagaactgccagaaaaagacagctggactgtcaatgatatacagaaattagtgggaaaactaaattgggcaagtcaaattt

SEQ ID NO: 3 - DNA, 159 bp, Artificial Sequence
  conserved sequence 1 on pol gene from HIV-1
atcaagctgaacaccttaagacagcagtacagatggcagtattcattcacaattttaaaagaaaaagggggattggggggtacagtgcaggggaaagaataatagacataatagcaacagacatacaaactaaagaattacaaaaacaaattacaaaaa

SEQ ID NO: 4 - DNA, 167 bp, Artificial Sequence
  conserved sequence 2 on pol gene from HIV-1
aaagaataatagacataatagcaacagacatacaaactaaagaattacaaaaacaaattacaaaaattcaaaattttcgggtttattacagggacagcagagacccaatttggaaaggaccagcaaaactactctggaaaggtgaaggggcagtagtaatacaagat

SEQ ID NO: 5 - DNA, 150 bp, Artificial Sequence
  conserved sequence 1 on ltr gene from HIV-1
ggactttccgccaaggactttccagagaggtgtagtctaggcaggactaaggagtggctagccctcagatgctgcatataagcagctgctttttgcctgtactaggtctctctaggtagaccagatctgagcctgggagctctctggcta

SEQ ID NO: 6 - DNA, 230 bp, Artificial Sequence
  conserved sequence 2 on ltr gene from HIV-1
agtggctaaccctcagatgctgcatataagcagccgcttttcgcttgtactgggtctctcttggtagaccagatagagcctgggagctctctggctagcaagggaacccactgcttaagcctcaataaagcttgccttgagtgcctaaagtagtgtgtgcccgtctgtgtgtgactctggtaactagagatccctcagacccttttagtcagtgtggaaaatctctagca

SEQ ID NO: 7 - DNA, 1718 bp, Artificial Sequence
  sequence of gene from phage MS2 (MS2 gene)
cattcctcggaggtttgccctgtgcgagcttttagtacgtttgacctgtgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccaaaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcatatgagatgcttacgaaggttcaccttcaagagtttcttcctatgagagccgtacgtcaggtcggtactaacatcaagttagatggccgtctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtcgaagacaacaaagaagttcaactctttatgtattg

SEQ ID NO: 8 - DNA, 37 bp, Artificial Sequence
  MS2-F forward primer (MS2-F)
catcaccacagccagcattcctcggaggtttgccctg

SEQ ID NO: 9 - DNA, 37 bp, Artificial Sequence
  MS2-R reverse primer (MS2-R)
cattatgcggccgcacaatacataaagagttgaactt

SEQ ID NO: 10 - DNA, 41 bp, Artificial Sequence
  HIV-F forward primer (HIV-F)
agaaggagatatacatatgttccctcaaatcactctttggc

SEQ ID NO: 11 - DNA, 38 bp, Artificial Sequence
  HIV-R reverse primer (HIV-R)
cagactcgagggtacctgctagagattttccacactga

(19) United States
(12) Patent Application Publication
(10) Pub. No.: US 2024/0084313 A1
(43) Pub. Date: Mar. 14, 2024

(54) HIV Pseudovirus Particles and Method for Preparing the Same

(71) Applicants: Chengdu BOE Optoelectronics Technology Co., Ltd., Chengdu, Sichuan (CN); BOE Technology Group Co., Ltd., Beijing (CN)

(72) Inventor: Hairong JING, Beijing (CN)

(21) Appl. No.: 18/272,355

(22) PCT Filed: Dec. 31, 2021
(86) PCT No.: PCT/CN2021/143731

§ 371 Date: Jul. 14, 2023

(51) Int. Cl. C12N 15/70; C07K 14/005; C12N 7/00; C12N 9/22

(52) U.S. Cl. CPC C12N 15/70; C07K 14/005; C12N 7/00; C12N 9/22; C12N 2740/16022

(57) ABSTRACT

HIV pseudovirus particles and method for preparing the same. The pseudovirus particles contain conserved sequences of gag, pol and LTR regions on genes from HIV-1 and a protein from phage MS2. The method comprises the following steps: designing and selecting target fragments and a sequence of gene from phage MS2; direct synthesizing both of them respectively; inserting the sequence of gene from phage MS2 into pETDuet-1 vector to obtain pETDuet-MS2 vector; inserting the target fragments into pETDuet-MS2 vector to obtain pETDuet-MS2-HIV recombinant expression vector. The sequence of gene from phage MS2 and target fragments respectively are located between any restriction sites behind the 3′ends of two T7 promoters of pseudovirus particles. The pseudovirus particles are encapsulated with a plurality of RNA conserved fragments from HIV-1 simultaneously, and have high yield and good stability. The method is fast, convenient, efficient and cost-effective.

### CROSS-REFERENCE TO RELATED APPLICATION

The present application is a U.S. National Phase Entry of International Application No. PCT/CN2021/143731 having an international filing date of Dec. 31, 2021. The above-identified application is hereby incorporated by reference.

### SEQUENCE LISTING

The text file Sequence-listing created Jul. 6, 2023, filed herewith, is hereby incorporated by reference.

### TECHNICAL FIELD

The present disclosure relates to the field of biomedicine, particularly to the field of genetic engineering, and specifically to pseudovirus particles comprising RNA fragments of human immunodeficiency virus (HIV) and a method for preparing the same.

### BACKGROUND

The pathogen of AIDS, i.e. acquired immunodeficiency syndrome is human immunodeficiency virus (HIV). HIV mainly invades the immune system of human body, and eventually leads to the deficiency of cellular immunity, which causes an occurrence of various opportunistic infections and tumors. At present, AIDS has become an important public health problem that seriously threatens public health.

### SUMMARY

The following is a summary of subject matters described herein in detail. The summary is not intended to limit the protection scope of claims.

An embodiment of the present disclosure provides pseudovirus particles comprising RNA fragments of human immunodeficiency virus (HIV).

An embodiment of the present disclosure provides a recombinant expression vector comprising conserved sequence fragments of gag1, gag2, pol1, pol2, LTR1, LTR2 regions on genes from HIV-1 and a sequence of gene from phage MS2.

In some exemplary embodiments, the sequence of gene from phage MS2 and conserved sequence fragments of gag1, gag2, pol1, pol2, LTR1 and LTR2 regions on genes from HIV-1 respectively are located between any restriction sites behind the 3′ends of two T7 promoters in the recombinant expression vector.

In some exemplary embodiments, the gag1 is the sequence set forth in SEQ ID NO: 1, the gag2 is the sequence set forth in SEQ ID NO: 2, the pol1 is the sequence set forth in SEQ ID NO: 3, the pol2 is the sequence set forth in SEQ ID NO: 4, the LTR1 is the sequence set forth in SEQ ID NO: 5, and the LTR2 is the sequence set forth in SEQ ID NO: 6.

In some exemplary embodiments, the sequence of gene from phage MS2 is the sequence set forth in SEQ ID NO: 7.

In some exemplary embodiments, the sequence of gene from phage MS2 and conserved sequence fragments of the gag1, gag2, pol1, pol2, LTR1, LTR2 regions on genes from HIV-1 are sequentially connected in direct.

An embodiment of the present disclosure also provides an expression vector comprising SEQ ID NOs: 1-7 as described above for expression in a host cell.

An embodiment of the present disclosure also provides pseudovirus particles, which is obtained by expressing the recombinant expression vector as described above or the expression vector as described above in a host cell; and
the pseudovirus particles comprise conserved sequence fragments of gag1, gag2, pol1, pol2, LTR1 and LTR2 regions on genes from HIV-1 and a protein from phage MS2.

In some exemplary embodiments, the resulting pseudovirus particles are present in the form of RNA of conserved regions such as gag pol and LTR of HIV packaged by the protein from phage MS2.

An embodiment of the present disclosure also provides a mixture comprising the recombinant expression vector as described above and the pseudovirus particles as described above,
or,
the expression vector as described above and the pseudovirus particles as described above.

An embodiment of the present disclosure also provides a method for preparing the pseudovirus particles as described above, the method comprising:
designing and selecting the target fragments and the sequence of gene from phage MS2;
directly synthesizing the target fragments and the sequence of gene from phage MS2 respectively;
inserting the sequence of gene from phage MS2 into a pETDuet-1 vector to obtain a pETDuet-MS2 vector;
inserting the target fragment into the pETDuet-MS2 vector to obtain a pETDuet-MS2-HIV recombinant expression vector.

In some exemplary embodiments, inserting the sequence of phage MS2 gene is performed using recombinant cloning techniques, with the pETDuet-1 vector being linearized by double digestion using endonucleases BamH I/Hind III, and the sequence of gene from phage MS2 being cloned into the pETDuet-1 vector using EasyGeno Assembly Cloning kit.

In some exemplary embodiments, inserting the target fragments is performed using recombinant cloning techniques, with the pETDuet-MS2 vector being linearized by double digestion using endonucleases EcoR V/Kpn I, and the target fragments being cloned into the pETDuet-MS2 vector using EasyGeno Assembly Cloning kit.

In some exemplary embodiments, the sequence of gene from phage MS2 and the target fragments respectively are located between any restriction sites behind the 3′ends of two T7 promoters in the PETDuet-MS2-HIV recombinant expression vector; and the sequence of phage MS2 gene and the target fragments are sequentially connected in direct, respectively.

Other aspects may be understood upon reading and understanding the drawings and detailed description.

### BRIEF DESCRIPTION OF DRAWINGS

Accompanying drawings are provided for further understanding of technical solutions of the present disclosure, and constitute a part of the specification, and together with the embodiments of the present disclosure, are used for explaining the technical schemes of the present disclosure, but do not constitute limitations on the technical schemes of the present disclosure. Shapes and sizes of one or more components in the drawings do not reflect true scales, and are only intended to schematically describe the contents of the present disclosure.

FIG. 1 is a flowchart of a method for preparing exemplary pseudovirus particles of the present disclosure.

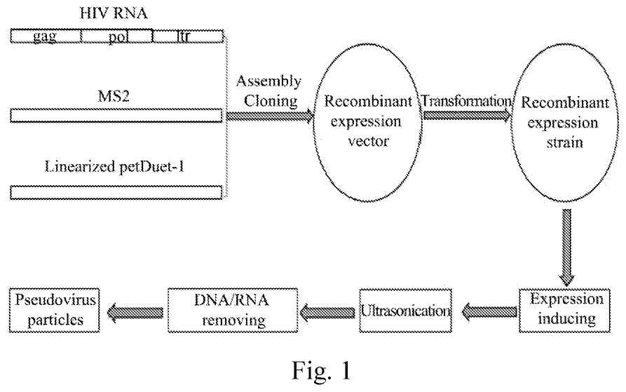

FIG. 2 shows the location where the target genes and the gene from MS2 are inserted into the pETDuet vector.

### DETAILED DESCRIPTION

To make the objectives, technical solutions, and advantages of the present disclosure clearer, the embodiments of the present disclosure will be described in detail below. It is to be noted that the embodiments in the present disclosure and features in the embodiments may be randomly combined with each other if there is no conflict.

The embodiments of the present disclosure will be described below in combination with the drawings in detail. Implementations may be implemented in a plurality of different forms. Those of ordinary skills in the art may easily understand such a fact that implementation modes and contents may be transformed into one or more forms without departing from the purpose and scope of the present disclosure. Therefore, the present disclosure should not be explained as being limited to the contents described in following implementation modes only. The embodiments in the present disclosure and features in the embodiments may be combined randomly with each other without conflict.

All technical and scientific terms used herein have the same meanings as understood by those of ordinary skill in the art to which the present disclosure pertains, unless otherwise defined. Although any methods and materials similar or equivalent to those described herein may be used to practice or test the present disclosure, preferred methods and materials are described. For the purposes of the present disclosure the following terms are defined hereinafter.

In this application, unless otherwise explicitly stated, the use of the singular includes the plural. It must be noted that the singular forms “a”, “an” and “the” as used in this specification include plural references, unless the context clearly dictated otherwise. In this application, unless otherwise stated, the use of “or” means “and/or”.

The terms “about” or “approximately” mean to be within an acceptable error range of a particular value, as determined by one of ordinary skill in the art, which will depend in part on how the value is measured or determined, i.e. the limitations of the measurement system. For example, “about” may mean to be within one or more than one standard deviations according to the practice in the art. Alternatively, “about” may mean a range of up to 20%, up to 10%, up to 5%, or up to 1% of a particular value. In other examples, an amount of “about 10” includes 10 and any amount from 9 to 11.

In yet other instances, the term “about” referring to a reference value may also include a range of values of plus or minus 10%, 9%, 8%, 7%, 6%, 5%, 4%, 3%, 2%, or 1% of that value. Alternatively, particularly in relation to biological systems or processes, the term “about” may mean to be within an order of magnitude of a value. When specific values are described in the present application and claims, the term “about” is assumed to mean to be within an acceptable margin of error for the specific values unless otherwise stated.

As use in this specification and one or more claims, the words “comprising” (and any form of comprising, such as “comprise” and “comprises”), “having” (and any form of having, such as “have” and “has”), “including” (and any form of including, such as “include” and “includes”) or “containing” (and any form of containing, such as “contains” and “contain”) are inclusive or open, and do not exclude additional, unlisted elements or method steps. It is contemplated that any of the embodiments discussed in this specification may be implemented with reference to any method or combination of the present disclosure and vice versa. In addition, the combination of the present disclosure may be used to implement the method of the present disclosure.

Vector refers to a kind of DNA molecule capable of self-replicating that transfers DNA fragments (target genes) to recipient cells in genetic engineering recombinant DNA technology. The target genes and the gene from MS2 of the present disclosure can be recombined with vector DNA (e.g. expression vectors) according to conventional techniques, which includes the EasyGeno Assembly Cloning kit and blunt-terminated or interlaced-terminated ends for ligation, restriction enzyme digestion to provide suitable ends, filling sticky ends where appropriate, alkaline phosphatase treatment to avoid undesired ligations, and ligation with suitable ligases. Techniques for these operations are disclosed in, for example, Maniatis et al., Molecular Cloning, Lab. Manual (Cold Spring Harbor Lab. Press, NY, 1982 and 1989), and Ausubel, 1987, 1993. Thus the present disclosure provides vectors or expression vectors comprising the target genes and the gene from phage MS2 listed herein.

As used herein, the phrase “sequentially connected in direct” refers to the direct synthesis of fragments in the foregoing order of the fragments using techniques known in the art. For example, conserved sequence fragments of gag1, gag2, pol1, pol2, LTR1, LTR2 regions on target genes from HIV-1 as described herein are sent to Sangon Biotech (Shanghai) Co., Ltd., for direct synthesis according to the above order. However, it is also within the scope of the present disclosure that conserved sequence fragments of the gag1, gag2, pol1, pol2, LTR1, LTR2 regions on genes from HIV-1 may be connected in other orders in other ways.

The target genes and the gene from phage MS2 of the present disclosure are placed in an expression vector and then they are transfected into host cells, such as Escherichia coli cells, to obtain pseudovirus particles in recombinant host cells. Many vectors are available. Vector components generally include, but are not limited to, one or more of the following: signal sequences, replication origin, one or more selection marker genes, enhancer elements, promoters, and transcription termination sequences.

### EXAMPLES

At present, there are various methods for the diagnosis and treatment of AIDS, among which nucleic acid load analysis, namely real-time fluorescence quantitative PCR, has been widely used for the diagnosis and supplementary diagnosis of AIDS patients, medication guidance and curative effect detection and evaluation. Commercial HIV nucleic acid detection kits have been developed by different manufacturers. Due to the consideration of biosafety, the raw materials for preparing calibrators and quality control products in most manufacturers' kits are pseudovirus particles assembled based on capsid protein of MS2 phage. Theoretically, pseudovirus particles are close to real viruses in appearance, have no biological infectivity, and can be obtained stably in large quantities. There are mainly following problems based on analysis of existing HIV pseudovirus particles: (1) the RNA sequence of HIV contained in the pseudovirus particles in a commercial kit is only within the detection range of primers and probes of its own manufacturer, that is, a positive result can be only obtained by detection using the reagents of its manufacturer, and the quality control products in different kits cannot be used interchangeably; (2) most of the RNA sequences contained in pseudovirus particles are less than 700 bp, and generally 300 bp-500 bp; the main reason is that there lacks technology to package long fragments of HIV RNA sequence at present, which leads to great differences in the analysis and detection results of various quantitative kits; (3) the preparation of pseudovirus involves complicated technical steps, which mainly adopt traditional enzyme digestion and ligation methods; (4) the preparation efficiency from expression vector to pseudovirus particles is not high, leading to low yield, low purity and poor stability of finished products, which seriously affects and limits the development of pseudovirus.

A solution to the above problems discloses a purification technology of pseudovirus particles. A 6His purified tag was added between position 15 and position 16 of amino acids of the capsid protein in MS2 phage, and then the RNA-protein complex pseudovirus particles were expressed, and then the pseudovirus particles were captured by protein purification methods, thus simplifying the preparation process of pseudovirus particles and improving the purification quality. Another solution discloses a method for preparing pseudovirus particles containing long chains of HIV RNA using plasmid co-transfection. Two kinds of cloning plasmids containing the sequence of MS2 gene and conserved sequences of gene from HIV were prepared respectively, and then pseudovirus particles were induced to express by double plasmid co-transfection technology. To a certain extent, the above methods or technologies are helpful to prepare pseudovirus particles containing long chains of HIV RNA with high purity, and provide certain guidance for solving the existing problems. However, these methods also have many shortcomings, such as the double plasmid co-transfection methods cannot ensure the same concentration of different plasmids in the same cell, which finally makes the MS2 capsid protein encapsulate RNA with low efficiency and poor stabilities of the pseudovirus particles obtained; meanwhile, the whole experimental process and operation are complicated, which is not conducive to the preparation of high-quality pseudovirus particles that meet the commercial application.

### Example 1. Selection of Target Fragments

1) Selecting the conserved sequences of gag, pol and LTR regions on genes from HIV-1, and selecting two conserved fragments for each region, as follows:

Seq-gag1 (SEQ ID NO: 1):
TTCCCTCAAATCACTCTTTGGCAACGACCCCTTGTCACAGTAAAAATAG
GAGGACAGCTGAAAGAAGCTCTATTAGATACAGGAGCAGATGATACAGT
ATTAGAAGATATAAATTTGCCAGGAAAATGGAAACCAAAAATGATAGGG
GGAATTGGAGGTTTTATCAAGGTAAA
Seq-gag2 (SEQ ID NO: 2):
GCATCAGAAGGAACCTCCATTCCTTTGGATGGGATATGAACTCCATCCT
GACAGATGGACAGTCCAGCCTATAGAACTGCCAGAAAAAGACAGCTGGA
CTGTCAATGATATACAGAAATTAGTGGGAAAACTAAATTGGGCAAGTCA
AATTT
Seq-pol1 (SEQ ID NO: 3):
ATCAAGCTGAACACCTTAAGACAGCAGTACAGATGGCAGTATTCATTCA
CAATTTTAAAAGAAAAAGGGGGATTGGGGGGTACAGTGCAGGGGAAAGA
ATAATAGACATAATAGCAACAGACATACAAACTAAAGAATTACAAAAAC
AAATTACAAAAA
Seq-pol2 (SEQ ID NO: 4):
AAAGAATAATAGACATAATAGCAACAGACATACAAACTAAAGAATTACA
AAAACAAATTACAAAAATTCAAAATTTTCGGGTTTATTACAGGGACAGC
AGAGACCCAATTTGGAAAGGACCAGCAAAACTACTCTGGAAAGGTGAAG
GGGCAGTAGTAATACAAGAT
Seq-ltr1 (SEQ ID NO: 5):
GGACTTTCCGCCAAGGACTTTCCAGAGAGGTGTAGTCTAGGCAGGACTA
AGGAGTGGCTAGCCCTCAGATGCTGCATATAAGCAGCTGCTTTTTGCCT
GTACTAGGTCTCTCTAGGTAGACCAGATCTGAGCCTGGGAGCTCTCTGG
CTA
Seq-ltr2 (SEQ ID NO: 6):
AGTGGCTAACCCTCAGATGCTGCATATAAGCAGCCGCTTTTCGCTTGTA
CTGGGTCTCTCTTGGTAGACCAGATAGAGCCTGGGAGCTCTCTGGCTAG
CAAGGGAACCCACTGCTTAAGCCTCAATAAAGCTTGCCTTGAGTGCCTA
AAGTAGTGTGTGCCCGTCTGTGTGTGACTCTGGTAACTAGAGATCCCTC
AGACCCTTTTAGTCAGTGTGGAAAATCTCTAGCA

The above six sequences of genes from HIV (SEQ ID NOs: 1-6) were sequentially connected in direct and sent to Sangon Biotech (Shanghai) Co., Ltd., for synthesis. However, it is also within the scope of the present disclosure that conserved sequence fragments of the gag1, gag2, pol1, pol2, LTR1, LTR2 regions on genes from HIV-1 may be connected in other orders in other ways.

2) the sequence of gene from phage MS2 (SEQ ID NO: 7)

Selecting the sequence of gene from phage MS2 from the NCBI, wherein the sequence of gene from phage MS2 was the gene sequence of capsid protein from phage MS2, specifically as follows:

CATTCCTCGGAGGTTTGCCCTGTGCGAGCTTTTAGTACGTTTGACCTGT
GCGAGCTTTTAGTACCCTTGATAGGGAGAACGAGACCTTCGTCCCCTCC
GTTCGCGTTTACGCGGACGGTGAGACTGAAGATAACTCATTCTCTTTAA
AATATCGTTCGAACTGGACTCCCGGTCGTTTTAACTCGACTGGGGCCAA
AACGAAACAGTGGCACTACCCCTCTCCGTATTCACGGGGGGCGTTAAGT
GTCACATCGATAGATCAAGGTGCCTACAAGCGAAGTGGGTCATCGTGGG
GTCGCCCGTACGAGGAGAAAGCCGGTTTCGGCTTCTCCCTCGACGCACG
CTCCTGCTACAGCCTCTTCCCTGTAAGCCAAAACTTGACTTACATCGAA
GTGCCGCAGAACGTTGCGAACCGGGCGTCGACCGAAGTCCTGCAAAAGG
TCACCCAGGGTAATTTTAACCTTGGTGTTGCTTTAGCAGAGGCCAGGTC
GACAGCCTCACAACTCGCGACGCAAACCATTGCGCTCGTGAAGGCGTAC
ACTGCCGCTCGTCGCGGTAATTGGCGCCAGGCGCTCCGCTACCTTGCCC
TAAACGAAGATCGAAAGTTTCGATCAAAACACGTGGCCGGCAGGTGGTT
GGAGTTGCAGTTCGGTTGGTTACCACTAATGAGTGATATCCAGGGTGCA
TATGAGATGCTTACGAAGGTTCACCTTCAAGAGTTTCTTCCTATGAGAG
CCGTACGTCAGGTCGGTACTAACATCAAGTTAGATGGCCGTCTGTCGTA
TCCAGCTGCAAACTTCCAGACAACGTGCAACATATCGCGACGTATCGTG
ATATGGTTTTACATAAACGATGCACGTTTGGCATGGTTGTCGTCTCTAG
GTATCTTGAACCCACTAGGTATAGTGTGGGAAAAGGTGCCTTTCTCATT
CGTTGTCGACTGGCTCCTACCTGTAGGTAACATGCTCGAGGGCCTTACG
GCCCCCGTGGGATGCTCCTACATGTCAGGAACAGTTACTGACGTAATAA
CGGGTGAGTCCATCATAAGCGTTGACGCTCCCTACGGGTGGACTGTGGA
GAGACAGGGCACTGCTAAGGCCCAAATCTCAGCCATGCATCGAGGGGTA
CAATCCGTATGGCCAACAACTGGCGCGTACGTAAAGTCTCCTTTCTCGA
TGGTCCATACCTTAGATGCGTTAGCATTAATCAGGCAACGGCTCTCTAG
ATAGAGCCCTCAACCGGAGTTTGAAGCATGGCTTCTAACTTTACTCAGT
TCGTTCTCGTCGACAATGGCGGAACTGGCGACGTGACTGTCGCCCCAAG
CAACTTCGCTAACGGGGTCGCTGAATGGATCAGCTCTAACTCGCGTTCA
CAGGCTTACAAAGTAACCTGTAGCGTTCGTCAGAGCTCTGCGCAGAATC
GCAAATACACCATCAAAGTCGAGGTGCCTAAAGTGGCAACCCAGACTGT
TGGTGGTGTAGAGCTTCCTGTAGCCGCATGGCGTTCGTACTTAAATATG
GAACTAACCATTCCAATTTTCGCTACGAATTCCGACTGCGAGCTTATTG
TTAAGGCAATGCAAGGTCTCCTAAAAGATGGAAACCCGATTCCCTCAGC
AATCGCAGCAAACTCCGGCATCTACTAATAGACGCCGGCCATTCAAACA
TGAGGATTACCCATGTCGAAGACAACAAAGAAGTTCAACTCTTTATGTA
TTG

The sequence of phage MS2 gene was sent to Sangon Biotech (Shanghai) Co., Ltd., for synthesis.

### Example 2. Construction of Recombinant pETDuet-MS2 Expression Strain

The phage MS2 gene was inserted into pETDuet-1 vector (Unibio) by recombinant cloning technology.

1) Linearization of pETDuet-1 Vector

BamH I/Hind III endonucleases (NEB) were used to linearize pETDuet-1 vector. The reaction system and conditions were shown in Table 1 below.

TABLE 1
BamH I/Hind III Enzyme Digestion System
BamH I/Hind III Enzyme Digestion System

| Components | amount (μL)/reaction |
|---|---|
| BamH I | 1 |
| Hind III | 1 |
| 10 × Buffer 3.1 | 5 |
| Plasmid | 5 |
| Ultrapure water | 38 |

The enzyme digestion reaction solution prepared above was put into a PCR instrument (LightCycler®96). After incubating at 37° C. for 1 h, the reaction products were subjected to gel electrophoresis and the linearized expression vector was recovered.

### 2) Acquisition of the Target Fragments to be Inserted

Primers were designed for amplification of phage MS2 gene fragment and target gene fragments, respectively

TABLE 2
Primers for amplification of phage MS2 gene fragment and target gene fragments

| Primer | | Amplification |
|---|---|---|
| names | Sequences | products |
| MS2-F | CATCACCACAGCCAGCATTCCTCGGAGGTTTGCCCTG (SEQ | MS2 |
| | ID NO: 8) | |
| MS2-R | CATTATGCGGCCGCACAATACATAAAGAGTTGAACTT (SEQ | |
| | ID NO: 9) | |
| HIV-F | AGAAGGAGATATACATATGTTCCCTCAAATCACTCTTTGGC | HIV |
| | (SEQ ID NO: 10) | |
| HIV-R | CAGACTCGAGGGTACCTGCTAGAGATTTTCCACACTGA | |
| | (SEQ ID NO: 11) | |

PCR amplification was carried out by using the above primers with artificially synthesized phage MS2 gene and target genes as templates, and the reaction system and conditions were as follows.

TABLE 3
Reaction system

| Components | amount (μL)/reaction |
|---|---|
| PrimeSTAR Max Premix (2×)(Takara | 20 |
| Biomedical Technology (Beijing) Co., Ltd) | |
| Upstream primers (10 μM) | 1 |
| Downstream primers (10 μM) | 1 |
| Templates | 1 |
| Water | 17 |

TABLE 4
PCR reaction conditions

| Steps | Temperature | Time | numbers of Cycles |
|---|---|---|---|
| Denaturation | 98° C. | 10 s | 35 |
| Annealing, extending | 68° C. | 30 s | |

After the reaction was completed, the PCR products were detected by gel electrophoresis, and the target bands were recovered.

### 3) Fragment Ligation

MS2 fragment was cloned into pETDuet-1 vector by EasyGeno Assembly Cloning kit (TIANGEN BIOTECH (BEIJING) Co., Ltd.). The reaction system and conditions were shown in Table 5 below.

TABLE 5
Reaction system and conditions

| Components | amount (μL)/reaction |
|---|---|
| Linearized vector | 3 |
| MS2 Product Recovered | 2 |
| 2 × EasyGeno Assembly Mixs | 5 |

The reaction was carried out at 37° C. for 30 min

### 4) Transformation

DH5α competent cells (Takara Biomedical Technology (Beijing) Co., Ltd) were thawed on ice, 10 μL of the previous ligation product was added into the thawed competent cells, gently blown and mixed, and placed on ice for 30 min, heat shocked at 42° C. for 90 seconds, and then placed on ice for 3 min. And then the transformation products were added into 890 μL SOC medium (Sangon Biotech (Shanghai) Co., Ltd.) for resuscitation at 37° C. for 1 h. After centrifuging at 6000 rpm for 30 s, the lower precipitates were plated on LB plate containing ampicillin (Beijing Solarbio Science & Technology Co., Ltd.), and incubated overnight at 37° C.

5) Acquisition of pETDuet-MS2 Plasmid

Monoclonal colonies were picked out, and the positive colonies identified were cultured for expansion, and then plasmids were extracted.

6) Construction of pETDuet-MS2-HIV Recombinant Expression Vector

The genes from HIV were inserted into pETDuet-MS2 vector by recombinant cloning technology.

\textcircled{1} Linearization of pETDuet-1 Vector

The pETDuet-MS2 vector was linearized with endonucleases EcoR V/Kpn I (NEB). The reaction system and conditions were shown in Table 6 below.

TABLE 6
Reaction system and conditions
EcoR V/Kpn I

| Components | amount (μL)/reaction |
|---|---|
| EcoR V | 1 |
| Kpn I | 1 |
| 10 × Buffer 3.1 | 5 |
| Plasmid | 5 |
| Ultrapure water | 38 |

The enzyme digestion reaction solution prepared above was put into a PCR instrument (LightCycler®96). After incubating at 37° C. for 1 h, the reaction products were subjected to gel electrophoresis and the linearized expression vector was recovered.

### \textcircled{2} Fragment Ligation

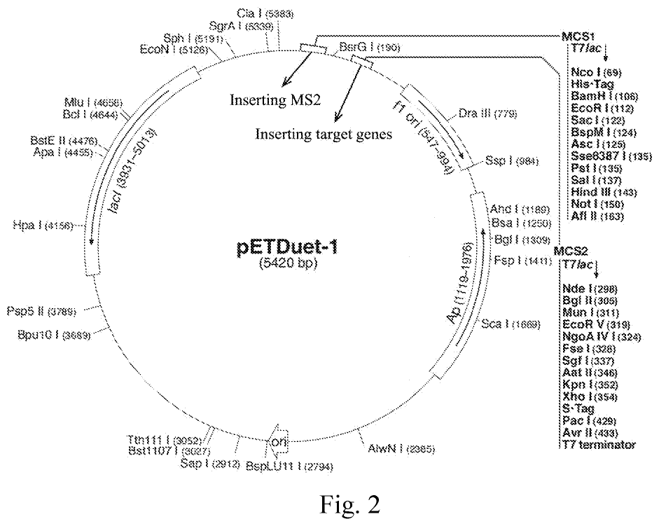

The fragments of the genes from HIV were cloned into PETDuet-MS2 vector (see FIG. 2), using EasyGeno Assembly Cloning kit. The reaction system and conditions were shown in Table 7 below.

TABLE 7
Reaction system and conditions

| Components | amount (μL)/reaction |
|---|---|
| Linearized vector | 3 |
| HIV products recovered | 2 |
| 2 × EasyGeno Assembly Mixs | 5 |

The reaction was carried out at 37° C. for 30 min

### \textcircled{3} Transformation

DH5α competent cells (Takara Biomedical Technology (Beijing) Co., Ltd) were thawed on ice, 10 μL of the previous ligation product was added into the thawed competent cells, gently blown and mixed, and placed on ice for 30 min, heat shocked at 42° C. for 90 seconds, and then placed on ice for 3 min. And then the transformation products were added into 890 μL SOC medium for resuscitation at 37° C. for 1 h. After centrifuging at 6000 rpm for 30 s, the lower precipitates were plated on LB plate containing ampicillin (Beijing Solarbio Science & Technology Co., Ltd.), and cultured overnight at 37° C.

\textcircled{4} Acquisition of pETDuet-MS2-HIV Plasmid

Monoclonal colonies were picked out, and the positive colonies identified were cultured for expansion, and then plasmids were extracted.

7) Construction of pETDuet-MS2-HIV Recombinant Expression Strain

BL21 (DE3) pLysS competent cells were thawed on ice, and 1 μL pETDuet-MS2-HIV plasmids were added into the thawed competent cells, which were gently beaten and mixed, and placed on ice for 30 min, heat shocked at 42° C. for 90 seconds, and then placed on ice for 3 min. And then the transformation products were added into 890 μL SOC medium for resuscitation at 37° C. for 1 h. After centrifuging at 6000 rpm for 30 s, the lower precipitates were plated on LB plate containing ampicillin (Beijing Solarbio Science & Technology Co., Ltd.), and cultured overnight at 37° C.

### Example 3. Acquisition of Virus-Like Particles Containing the Genes from HIV

1. Induced Expression of Virus-Like Particles Containing the Genes from HIV

The recombinant strains identified as positive were added into 5 mL liquid LB medium containing ampicillin and incubated at 37° C. overnight with shaking at 180 rpm for activation. On the second day, 1 mL of culture medium was added into the liquid medium containing ampicillin+chloramphenicol in a ratio of 1:100, and cultured at 37° C. for 3-4 h with shaking at 180 rpm. When the OD600 was 0.8-1.0, 1 mL of IPTG 50 mg/mL was added into the bacteria solution, and cultured at 37° C. overnight with shaking at 180 rpm.

2. Purification of Virus-Like Particles Containing the Genes from HIV

1) the above bacteria solution was collected, centrifuged at 10000 rpm for 10 min, and the precipitated bacteria was obtained. The precipitated bacteria was washed with 20 mM PBS to remove the residual culture medium, and then the bacteria was re-suspended with 20 mM PBS. The bacteria was crushed using ultrasonic cell crusher. The reaction conditions were: ultrasonication for 2 s, stop for 9 s, and the total time was 30 min.

2) the ultrasonicated solution was centrifuged at 10000 rpm for 10 min, the supernatant was transferred to a new centrifuge tube, to which sodium chloride was added to a final concentration of 1M, and chloroform was added in equal volume at the same time; after fully reversed up and down, the tube was centrifuged at 10000 rpm for 10 min, the supernatant was transferred to a new centrifuge tube, to which PEG8000 is added to a final concentration of 20% under fully shaking and mixing, so that PEG8000 was completely dissolved.

3) the completely dissolved solution was incubated on ice for 1 h, centrifuged at 10000 rpm for 10 min, and the supernatant was discarded, the residual liquid was sucked with dust-free paper, 10 mL 20 mM PBS was added to re-suspend the precipitate, which was fully shaked and mixed thoroughly to ensure a complete dissolution.

4) The amount of DNAseI (QIAGEN) and Rnase A (QIAGEN), the catalyst and the amount thereof and the reaction conditions were optimized to eliminate free DNA and RNA in pseudovirus particle solution. The amount of DNAse I used was 200 U/10 mL, the amount of Rnase A used was 50 U/10 mL, and the amount of catalyst magnesium chloride (20 mM) (Sigma) was 1 mL/10 mL. The reaction conditions were incubation at 37° C. for 5-10 h. After the reaction was completed, an equal volume of chloroform (Sigma) was added, and centrifuged at 10000 rpm for 20 min to remove miscellaneous proteins, and the supernatant was obtained. This step was repeated twice, and the obtained supernatant contained the purified pseudovirus particles. The resulting pseudovirus particles were present in the form of RNA of conserved regions such as gag, pol and LTR of HIV packaged by the protein from phage MS2.

5) Verification of concentration and purity of pseudovirus particle stock solution

The RNA from purified pseudovirus was extracted by QIAGEN QIAamp Viral RNA Mini Kit (QIAGEN), and the reaction systems containing MMLV+Taq enzyme and Taq enzyme alone were prepared, and the nucleic acid was amplified at the same time.

TABLE 8
System and ΔCt value

| RNA system | DNA system | ΔCt |
|---|---|---|
| 5.64 | 20.23 | 14.59 |
| 5.25 | 20.14 | 14.89 |
| 5.86 | 20.36 | 14.50 |

| | |
|---|---|
| Average DNA residue | 1/25888 |

The results showed that the Ct value of the prepared pseudovirus stock solution was smaller (≤6), and the DNA residue was smaller, that is, the complete protein particles packaged were predominant in the solution of the pseudovirus particles, which indicated that the yield and packaging efficiency of the pseudovirus particles prepared by this method were high. By calculation, the pseudovirus residue was lower (less than 1/10000), and the purity was higher, which was calculated as follows: purity=1/2^{CtD-CtR}. Noting: CtD denotes CT value of DNA system and CtR denotes CT value of RNA system.

### Example 4. Stability of Pseudovirus Particles

The pseudovirus particles were stored in diluted storage solution containing trehalose (1%-5%) (Sigma), BSA (0.005%-0.04%) (Sigma), glycerol (1%-20%) and PBS (2 mM-20 mM) (Sigma).
1) Pseudovirus preservation solution containing 2% trehalose, 0.01% BSA, 10% glycerol and 10 mM PBS was prepared.
2) The pseudovirus particles obtained in Example 3 were sequentially diluted to a Ct value of around 30 using the pseudovirus preservation solution.
3) The pseudovirus particles obtained in step 2) were tested for stability under the conditions of 37° C. for 3 days or 2° C.-8° C. for 30 days and freezing and thawing was repeated for 10 times, and the stability was compared with stability of the pseudovirus particles stored at −20° C., and the difference between them was observed.

TABLE 9
Results of stability of pseudovirus particles stored at 37° C.
Stability at 37° C. for 3-day

| Test | Control | ΔCt |
|---|---|---|
| 30.23 | 29.86 | 0.37 |
| 30.15 | 30.01 | 0.14 |
| 30.59 | 30.12 | 0.47 |

TABLE 10
Results of stability of Pseudovirus particles stored at 2° C.-8° C.
Stability at 2° C.-8° C.

| 2° C.-8° C. | 2° C.-8° C. | 2° C.-8° C. | |
|---|---|---|---|
| for 10 days | for 20 days | for 30 days | Control |
| 29.99 | 29.88 | 30.25 | 29.86 |
| 30.05 | 30.12 | 30.44 | 30.01 |
| 30.56 | 30.44 | 30.25 | 30.12 |

TABLE 11
Results of stability of pseudovirus particles after repeated freeze-thaw
Stability of Repeated freeze-thaw

| 1 time | 4 times | 7 times | 10 times |
|---|---|---|---|
| 29.86 | 29.97 | 30.14 | 30.25 |
| 30.01 | 30.14 | 30.47 | 3036 |
| 30.12 | 30.25 | 30.47 | 30.45 |

The difference between the Ct values of the prepared pseudovirus in pseudovirus preservation solution at 37° C. for 3 days, 2° C.-8° C. for 30 days or repeated freezing and thawing for 10 times and the controls was less than 1, which indicated that the pseudovirus particles were stable under these conditions.

1. A recombinant expression vector, comprising conserved sequence fragments of gag1, gag2, pol1, pol2, LTR1, LTR2 regions on genes from HIV-1 and a sequence of gene from phage MS2.

2. The recombinant expression vector according to claim 1, wherein the sequence of gene from phage MS2 and the conserved sequence fragments of gag1, gag2, pol1, pol2, LTR1 and LTR2 regions on genes from HIV-1 respectively are located between any restriction sites behind the 3′ends of two T7 promoters in the recombinant expression vector.

3. The recombinant expression vector according to claim 1, wherein the gag1 is the sequence set forth in SEQ ID NO: 1, the gag2 is the sequence set forth in SEQ ID NO: 2, the pol1 is the sequence set forth in SEQ ID NO: 3, the pol2 is the sequence set forth in SEQ ID NO: 4, the LTR1 is the sequence set forth in SEQ ID NO: 5, and the LTR2 is the sequence set forth in SEQ ID NO: 6.

4. The recombinant expression vector according to claim 1, wherein the sequence of gene from phage MS2 is SEQ ID NO: 7.

5. The recombinant expression vector according to claim 1, wherein the sequence of gene from phage MS2 and the conserved sequence fragments of the gag1, gag2, pol1, pol2, LTR1, LTR2 regions on genes from HIV-1 are sequentially connected in direct.

6. An expression vector, comprising the sequences of SEQ ID NOs: 1-7 of the recombinant expression vector according to claim 3, for expression in a host cell.

7. Pseudovirus particles obtained by expressing the recombinant expression vector according to claim 1; and
the pseudovirus particles comprise conserved sequence fragments of gag1, gag2, pol1, pol2, LTR1 and LTR2 regions on genes from HIV-1 and a protein from phage MS2.

8. A mixture, comprising a recombinant expression vector the pseudovirus particles according to claim 7, wherein the recombinant expression vector comprises conserved sequence fragments of gag1, gag2, pol1, pol2, LTR1, LTR2 regions on genes from HIV-1 and a sequence of gene from phage MS2.

9. A method for preparing the pseudovirus particles of claim 7, the method comprising:
designing and selecting target fragments and the sequence of gene from phage MS2;
directly synthesizing the target fragments and the sequence of gene from phage MS2 respectively;
inserting the sequence of gene from phage MS2 into a pETDuet-1 vector to obtain a pETDuet-MS2 vector;
inserting the target fragments into the pETDuet-MS2 vector to obtain a pETDuet-MS2-HIV recombinant expression vector.

10. The method according to claim 9, wherein inserting the sequence of gene of phage MS2 is performed using recombinant cloning techniques, with the pETDuet-1 vector being linearized by double digestion using endonucleases BamH I/Hind III, and the sequence of gene from phage MS2 being cloned into the pETDuet-1 vector using EasyGeno Assembly Cloning kit.

11. The method according to claim 9, wherein inserting the target fragments is performed using recombinant cloning techniques, with the pETDuet-MS2 vector being linearized by double digestion using endonucleases EcoR V/Kpn I, and the target fragments being cloned into the pETDuet-MS2 vector using EasyGeno Assembly Cloning kit.

12. The method according to claim 9, wherein the sequence of gene from phage MS2 and the target fragments respectively are located between any restriction sites behind the 3′ends of two T7 promoters in the PETDuet-MS2-HIV recombinant expression vector; and the sequence of gene of phage MS2 and the target fragments are sequentially connected in direct.

13. Pseudovirus particles obtained by expressing the expression vector according to claim 6 in a host cell; and
the pseudovirus particles comprise conserved sequence fragments of gag1, gag2, pol1, pol2, LTR1 and LTR2 regions on genes from HIV-1 and a protein from phage MS2.
